(19) United States
(12) Patent Application Publication
(10) Pub. No.: US 2022/0400377 A1
(43) Pub. Date: Dec. 15, 2022

(54) Securing communication between a wireless data communication accessory for a drug delivery device and an external device

(71) Applicant: Sanofi, Paris (FR)

(72) Inventor: Stefan Alt, Frankfurt am Main (DE)

(21) Appl. No.: 17/776,312

(22) PCT Filed: Nov. 11, 2020
(86) PCT No.: PCT/EP2020/081786

§ 371 Date: May 12, 2022

(30) Foreign Application Priority Data
Nov. 14, 2019 (EP) 19306467.2
Apr. 9, 2020 (EP) 20315139.4

(51) Int. Cl. H04W 12/037; A61M 5/315; H04B 5/00; H04W 76/10; G06F 21/60; H04W 12/50

(52) U.S. Cl. CPC H04W 12/037; A61M 5/3155; H04B 5/0062; H04W 76/10; G06F 21/606; H04W 12/50; A61M 2205/3584; A61M 2205/8206; A61M 2205/3569

(57) ABSTRACT

A system comprising a wireless data communication accessory and a drug delivery device is disclosed, wherein the wireless data communication accessory is configured for data exchange with the drug delivery device and to secure data from the drug delivery device using cryptographic information assigned to the drug delivery device.

### CROSS REFERENCE TO RELATED APPLICATIONS

The present application is the national stage entry of International Patent Application No. PCT/EP2020/081786, filed on Nov. 11, 2020, and claims priority to Application No. EP 20315139.4, filed on Apr. 9, 2020, and Application No. EP 19306467.2, filed on Nov. 14, 2019, the disclosures of which are incorporated herein by reference.

### TECHNICAL FIELD

The present disclosure relates to securing communication between a wireless data communication accessory for a drug delivery device and an external device.

### BACKGROUND

A variety of diseases exists that require regular treatment by delivery, particularly injection of a medicament. Such injection can be performed by using injection devices, which are applied either by medical personnel or by patients themselves.

Drug injection devices particularly for usage by patients themselves may be equipped with electronics for measuring and storing data related to the usage. The usage related data may also be transmitted via a wireless link or a wireline connection to an external device such as a smartphone, a tablet or laptop computer, or in the cloud. For example, US 2019/0134305 A1 discloses a medication delivery device, for example an injection pen or a wearable pump, which can be paired with an external device for providing data captured from a flow sensor relating to medicine delivery to a patient to a paired external device. The device can have Bluetooth® communication and/or near field communication (NFC) circuits for proximity-based pairing and connectivity with the external device for real-time or deferred transfer of captured data to the external device.

### SUMMARY

In one aspect the present disclosure provides a system comprising a wireless data communication accessory and a drug delivery device, wherein the wireless data communication accessory is configured for data exchange with the drug delivery device and to secure data from the drug delivery device using cryptographic information assigned to the drug delivery device. The securing of the data from the drug delivery device may particularly mean encrypting the data, particularly patient related data such as data related to ejected dosages of a certain medicament. This allows to “bind” data captured with the drug delivery device and/or the wireless data communication accessory such as dosages and/or patient related data to a specific drug delivery device. When the wireless data communication accessory is used with different drug delivery devices, this may prevent a drug mix-up in the accessory, when data from the different drug delivery devices is stored in an internal storage of the accessory, and/or in an external device receiving data from the different drug delivery devices via the wireless data communication accessory. Additionally, with each new drug delivery device, the cryptographic information assigned to that specific drug delivery device may be used for securing the data so that security may be increased since not the same cryptographic information is used for different drug delivery devices. In other words, the cryptographic information is changed with each drug delivery device.

In embodiments, the wireless data communication accessory may be configured for communicating with an external device via a wireless communication and to secure the wireless communication for transmitting the data from the drug delivery device using the cryptographic information assigned to the drug delivery device. Thus, it may be ensured that only an authorized external device can receive the data from the drug delivery device.

In embodiments, the wireless data communication accessory may be configured to secure the data exchange between the accessory and the drug delivery device using the cryptographic information assigned to the drug delivery device. This may add a further security to the system since the entire transmission from data stored in the drug delivery device to the external device may be secured. Particularly, when the data exchange between the accessory and the drug delivery device is not proximity based, this may be helpful to avoid that remote devices can get unauthorized access to the data from the drug delivery device.

In embodiments, at least a part of the cryptographic information may be stored in a storage of the drug delivery device and readable by the accessory and/or the external device. Particularly, the drug delivery device may comprise passive short-range wireless communication means comprising the storage storing the cryptographic information, for example a NFC (Near Field Communication) tag integrated in or a NFC label attached to the drug delivery device.

In further embodiments, at least a part of the cryptographic information may be visibly applied to the drug delivery device, particularly in form of a machine readable code, for example a QR code, and/or at least a part of the cryptographic information may be stored in a storage of the external device, and/or at least a part of the cryptographic information may be stored on a server for downloading, particularly on a server of a cloud storage.

Particularly, when the cryptographic information comprise a two factor encryption method for example with a public and private key, the accessory could be used only as a kind of “data bridge”, and data transmitted from the drug delivery device to the external device via the accessory could be secured with an end-to-end encryption between the drug delivery device and the external device. The at least a part of the cryptographic information could for example comprise the public key assigned to the drug delivery device. The external device could then use the public key assigned to the drug delivery device to establish an end-to-end encryption with the drug delivery device. The public key may be for example visibly applied to the drug delivery device, stored in a storage of the external device and/or on a server for downloading.

In embodiments, the data exchange between the accessory and the drug delivery device may require a pairing of the accessory with the drug delivery device, wherein the cryptographic information may be required for the pairing. The pairing may be particularly provided for drug delivery devices comprising a power supply like a battery and electronics such as for example a Bluetooth® module. However, in principle a pairing may be also provided for drug delivery devices comprising only passive electronics such as a passive NFC tag. Then, the pairing may comprise for example a modification of at least a part of the cryptographic information stored in a storage of the passive electronics with the accessory, particularly when the accessory is used the first time with the drug delivery device, for example to avoid that the drug delivery device can be paired with another accessory and/or that the cryptographic information is used by another device and/or accessory. This may further increase security.

In embodiments, the drug delivery device and/or the accessory may comprise a storage for storing data related to selected and delivered drug dosages, and wherein the drug delivery device and/or the accessory is/are configured to protect the stored data related to selected and delivered drug dosages by means of the cryptographic information.

In a further aspect the present disclosure provides a wireless data communication accessory configured for usage with the system as disclosed and described herein and comprising an interface for data exchange between the accessory and a drug delivery device, wherein the accessory is configured to secure data from the drug delivery device using cryptographic information assigned to the drug delivery device.

In embodiments, the accessory may be configured for communicating with an external device via a wireless communication and to secure the wireless communication for transmitting the data from the drug delivery device using the cryptographic information assigned to the drug delivery device.

In embodiments, the interface for data exchange between the accessory and the drug delivery device may be configured to secure the data exchange using the cryptographic information assigned to the drug delivery device.

In a further embodiment, the accessory may be configured to read the cryptographic information from a storage of the drug delivery device, wherein the accessory may particularly comprise second wireless communication means configured for generating a radio frequency field for powering passive short-range wireless communication means of the drug delivery device comprising the storage storing the cryptographic information and receiving the cryptographic information from the storage of the passive short-range wireless communication means via short-range communication.

In a yet further aspect the present disclosure provides a drug delivery device provided for usage with the system as disclosed and described herein and comprising assigned cryptographic information being stored in a storage of the drug delivery device and/or being visibly applied to the drug delivery device, particularly in form of a machine readable code.

In a still further aspect the present disclosure provides a method for securing data from a drug delivery device, comprising providing a wireless data communication accessory, configuring the accessory for data exchange with the drug delivery device, obtaining cryptographic information assigned to the drug delivery device, reading data from a storage of the drug delivery, and securing the the read data using the obtained cryptographic information assigned to the drug delivery device.

In embodiments, the method may further comprise pairing the accessory with an external device, configuring the accessory for communicating with the paired external device, securing the communication with the paired external device using the obtained cryptographic information assigned to the drug delivery device, and transmitting the read data by means of the secured communication to the paired external device.

### BRIEF DESCRIPTION OF THE FIGURES

FIG. 1 shows a first embodiment of a system comprising a drug delivery device, a wireless data communication accessory, and external devices communicating with the wireless data communication accessory;

FIG. 2 shows a block diagram of an embodiment of the accessory and of the drug delivery device;

FIG. 3 shows the first embodiment of the accessory in different views;

FIG. 4 shows a second embodiment of the accessory in different views; and

FIGS. 5 to 7 show a third embodiment the accessory in different views.

### DETAILED DESCRIPTION

In the following, embodiments of the present disclosure will be described with reference to injection devices, particularly an injection device in the form of a pen. The present disclosure is however not limited to such application and may equally well be deployed with other types of drug delivery devices, particularly with another shape than a pen.

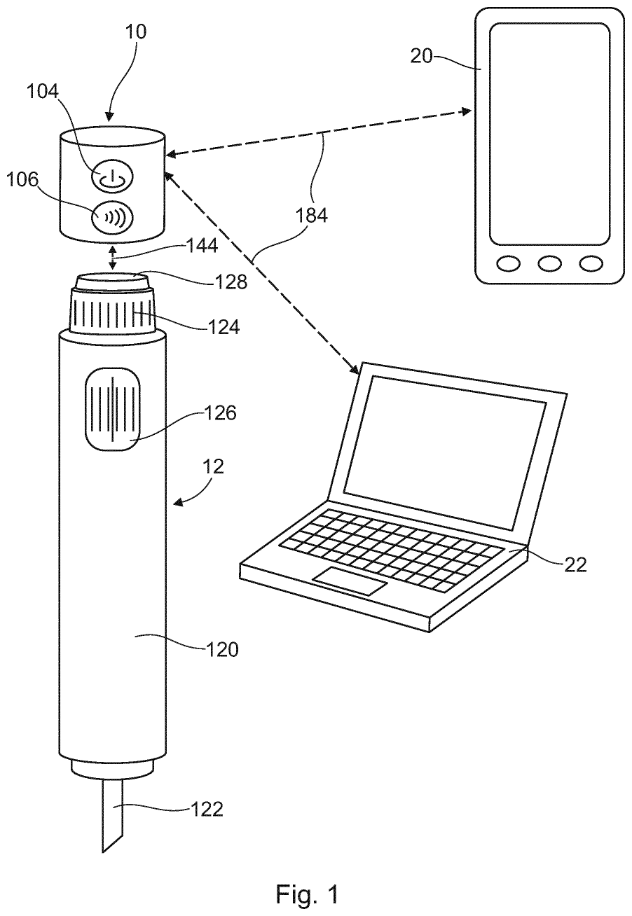

FIG. 1 shows a system comprising a drug delivery device 12 in the shape of an insulin injection pen, a wireless data communication accessory 10 attachable to the device 12, and an external device such as a smartphone 20 or a laptop computer 22.

The device 12 comprises an elongated body 120 having a pen-like shaped form for holding a drug cartridge and a dosage selection and delivery mechanism. At the lower end of the body 120, a syringe 122 for expelling a drug dosage and injecting this dosage in a patient's body is provided. The body 120 comprises at its other, upper end a dial knob 124 for selecting a drug dosage and an injection knob 128 for delivery of a selected dosage. A user of the device 12 selects a dosage by rotating the dial knob 124 around the longitudinal axis of the body 120. The selected dosage is shown on a display 126 integrated in the body 120. After dosage selection, the user may press the injection knob 128 in the direction of the longitudinal axis for expelling the selected dosage via the syringe 122 into a patient's body. The dosage selection and delivery mechanism contained in the body may comprises electronics (not shown) for detecting and for storing and transmitting selected and delivered dosages.

The wireless data communication accessory 10 can be attached to the device 12 by clipping it on the dial knob 124. The accessory 10 houses electronics (not shown) comprising first wireless communication means for establishing a first communication link 184 with an external device such as a smartphone 20 or a laptop computer 22, which may be paired with the wireless communication means, and comprising second wireless communication means and/or wired communication means for establishing a second communication link 144 for exchanging data with a data exchange interface of the device 12.

Different options for securing data from the drug delivery device 12, particularly dose related data, may be provided as will be described in hereinafter. Securing the data from the drug delivery 12 must be understood in the context of this disclosure in a broad sense, and particularly comprises securing a data access, data transmission and data storage. Particularly, the accessory 10 may be configured to secure data from the drug delivery device 12 in at least one of the following cases: when receiving the data from the drug delivery device 12; when storing the data from the drug delivery device 12 in an internal storage of the accessory 10; when transmitting the data from the drug delivery device 12 to an external device 20, 22; granting access to the data from the drug delivery device 12 stored in an internal storage of the accessory 10.

The first wireless communication link 184 may be secured using cryptographic information assigned to the drug delivery device 12. Particularly, the term “paired” may mean that the accessory 10 and the external devices 20, 22 share some secret data such as one or more cryptographic keys for establishing and/or securing data exchange via the first communication link 184.

In addition or alternatively, the second communication link 144 may be secured using cryptographic information assigned to the drug delivery device 12.

The accessory 10 may comprise a power button 104 for activating and deactivating power supply of the electronics manually. It may further comprise a wireless transmission button 106 provided for initiating a wireless transmission of data from the accessory to the external device 20 and/or 22. The button 106 may be also provided for initiating a pairing process of the accessory's 10 first wireless communication means with an external device 20, 22, for example by pressing the button 106 for a certain time period such as several seconds, thus switching the first wireless communication means in a pairing mode. The first wireless communication means can also be configured to establish automatically a communication link 184 with an already paired external device 20, 22 once the electronics of the accessory 10 is powered and the external device 20, 22 is within a range of maximum communication of the first wireless communication means.

In embodiments, activating the power supply of the electronics of the accessory 10 may be performed automatically, i.e. without manually pressing the button 104, for example by means of an integrated switch of the accessory, which may be activated when the accessory is attached to the device 12 or when a dosage is selected and/or delivered with the device 12. The integrated switch may be for example a mechanical switch or a magnetic switch, which may be activated when the accessory 10 is clipped on the dial knob 124 of the device 12 and/or when the dosage selection and delivery mechanism is used by turning the dial knob 124 and/or pressing the injection knob 128.

The first communication means may be configured for establishing a long-range wireless communication via radio frequency communication such as a Bluetooth® communication link 184 and/or a Wi-Fi™ direct communication link based on the IEEE 802.11 standard (ISO/IEC 8802-11) with the external devices 20, 22 over a distance of at least several centimetres, particularly at least one meter, and more particularly several meters. The maximum distance provided for communication may depend on the power supply requirements of the accessory 10. For example, when the accessory 10 is powered by a one-time usable battery, which should last several months, at least a year or even longer, the maximum distance may be configured by reducing the power requirements of the first communication means to meet the desired battery lifetime.

The second wireless communication means may be configured to establish a short-range wireless communication employing electromagnetic induction for data transmission such as a short-range data communication technology based on a RFID (Radio Frequency Identification) standard such as NFC.

For securing communication via the first wireless communication link 184, cryptographic information assigned to the drug delivery device 12 may be used. The cryptographic information may for example comprise some secret data such as one or more cryptographic keys, for example a symmetric encryption key or a public-private-key pair for an asymmetric encryption.

At least a part of the cryptographic information may be stored electronically for example in a storage of the data exchange interface of the device 12, and may be transmitted to the accessory 10 via the second communication link 144. At least a part of the cryptographic information may comprise for example a key of a public-private-key pair, which may be used to establish an asymmetric encryption, or a shared cryptographic key, which may be used to establish a symmetric encryption.

The at least a part of the cryptographic information may particularly be stored in a NFC tag integrated in the body 120 or attached to the body 120. This makes a contactless reading possible, for example with a NFC reader comprised by the accessory 10 or by an external device 20, 22. Alternatively or additionally, contacts may be provided at the device 12, which allow to read the at least a part of the cryptographic information when respective contacts of for example the accessory 10 are brought into electrical contact with these contacts and a reader circuitry comprised by the accessory 10 could then initiate a data exchange between the accessory 10 and the device 12.

Thus, when the accessory 10 is attached to or brought into close proximity of the device 12, and the communication link 144 is established, the accessory 10 may read from a storage of the device 12 data related to the device 12, which may contain the at least a part of the cryptographic information, particularly a key of a public-private-key pair or a shared cryptographic key. The accessory 10 may internally store the read cryptographic information and use the stored information for securing the wireless communication 184 with the external devices 20, 22 and/or for securing the second communication link 144 with the device 12. Additionally, the read cryptographic information may also be used by the accessory 10 to encrypt stored data, which were received from the device 12, particularly if these data relate to dosages expelled with the device 12 and are patient-related.

The at least a part of the cryptographic information may additionally or alternatively be visibly applied to the drug delivery device 12, for example printed on the elongated body 120. The form of the visible application may be machine and/or human readable. For example, a QR code may be applied to the body 120 containing the at least a part of the cryptographic information. The QR code may for example comprise a key of a public-private-key pair or a shared cryptographic key. It would be also possible to readably print an encryption key on the body 120, for example either a key of a public-private-key pair or a shared cryptographic key. The visibly applied cryptographic information may be obtained by using optical capturing means of the external devices 20, 22, particularly by using a camera device integrated in the devices 20, 22. A software such as an app can then configure the devices 20, 22 after a respective user input to activate the camera for capturing the visible cryptographic information, may process the captured information for example by some optical character recognition to obtain the cryptographic information, and store the so obtained cryptographic information for securing the wireless communication link 184 with the accessory 10. A further option may be that a user may read the cryptographic information visibly applied to the device 12, for example an encryption key printed on the body 120, and manually enters the cryptographic information into the devices 20, 22. When the cryptographic information is obtained by devices 20, 22, it may be transmitted to the accessory 10, which may be accomplished with a kind of secure key exchange procedure.

Instead of or additionally to applying the at least a part of the cryptographic information visibly to the drug delivery device 12, it may be printed on a packaging of the drug delivery device 12 and/or an IFU (Instructions for Use).

A further option may consist in storing the at least a part of the cryptographic information in a storage of the external device 20, 22. This may particularly comprise downloading the at least a part of cryptographic information, for example a key of a public-private-key pair or a shared cryptographic key, from a server computer. For example, a server computer remotely accessible may be provided by a distributor and/or manufacturer of the drug delivery device, and a patient may access the server computer, enter a certain code related to a specific drug delivery device owned by the patient to obtain cryptographic information assigned to the specific drug delivery device from the server computer, particularly a public kay or a shared cryptographic key, and store the obtained cryptographic information on the external device 20, 22, from which the server computer was accessed. The server computer may be part of a cloud storage.

A yet further option may consist in that the device 12 and/or accessory 10 encrypt data from the device 12 with a key of a public-private-key pair, and the private key may be visibly applied to the drug delivery device 12 as describe above and/or stored on a server particularly of a cloud storage for downloading in a storage of the external device 20, 22. Thus, a decryption of the encrypted data requires the private key.

A pairing of the accessory 10 and drug delivery device 12 may be required for data exchange. The pairing may for example comprise receiving and storing some identification information from the drug delivery device 12 in a storage of the accessory 10, which for example may be used to tag data received from the drug delivery device 12 and/or to ensure that only data from paired devices 12 are read by the accessory. The pairing may require the cryptographic information or at least part of the cryptographic information. For example, the accessory 10 may first read the cryptographic information from a storage of the device 12, and then pair itself with the device 12 using the read cryptographic information, or the accessory 10 may receive the cryptographic information from one of the external devices 20, 22, with which it may communicate, and then use the so received cryptographic information to pair itself with the drug delivery device 12. The pairing may also comprise that the accessory 10 or an external device 20, 22 may modify the cryptographic information read from the storage of the device 12. For example, when the cryptographic information is stored in a writeable storage of a NFC tag of the device 12, the accessory 10 or an external device 20, 22 may after reading out the cryptographic information from the storage of the NFC tag, modify the stored cryptographic information so that no other accessory 10 and/or external device 20, 22 can obtain the cryptographic information.

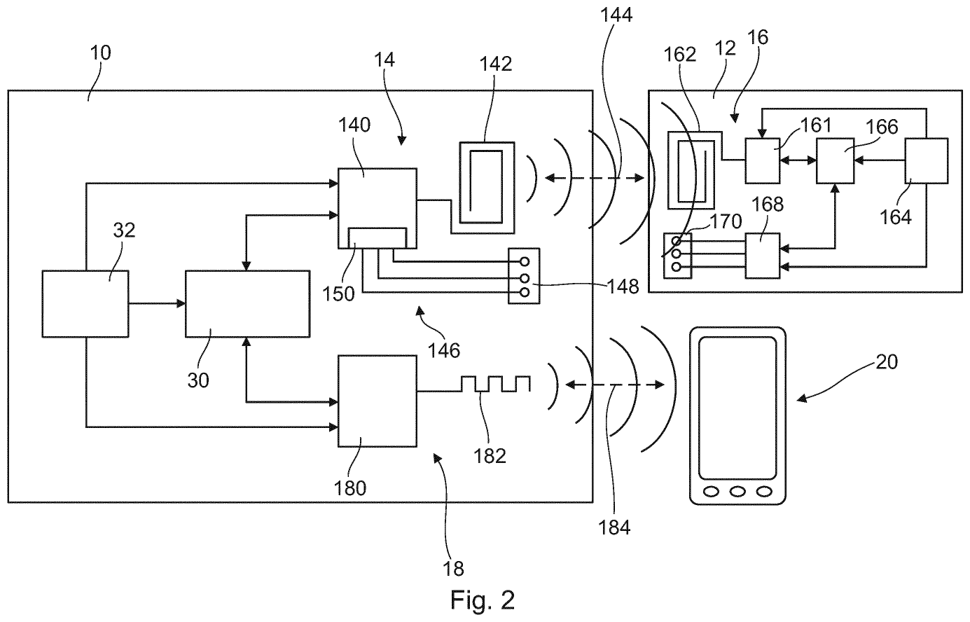

FIG. 2 shows an embodiment of the electronics of the accessory 10 and of the electronics of the drug delivery device 12 as block diagram.

The electronics of the accessory 10 comprises a controller 30, a battery 32, the first wireless communication means 18, and an interface 14 for data exchange with the electronics of the drug delivery device 12, particularly for receiving data related to drug selection and delivery being stored in an internal memory of the drug delivery device 12.

The first wireless communication means 18 comprises a transceiver circuitry 180, for example a Bluetooth® and/or a WiFi™ communication circuitry, and an antenna 182, for example a Bluetooth® and/or a WiFi™ antenna. The transceiver circuitry 180 may be particularly configured to communicate according to the Bluetooth® low energy (BLE) technology.

The interface 14 comprises second wireless communication means, particularly NFC reader circuitry 140 with an NFC loop antenna 142, and/or wired communication means 146 comprising at least one contact 148, particularly a sprung contact, and a serial communication interface circuitry 150 such as an UART/I2C/SPI/1-wire interface circuitry.

The controller 30 is coupled to the interface 14 and the first wireless communication means 18 and may be configured to control operation of these units such that data, which is received from battery 164 powered electronics 160 of the drug delivery device 12 via a short range communication between the second wireless communication means 140, 142 and short-range wireless communication means 16 of the drug delivery device 12, is processed to be transmitted via the first wireless communication means 18 to an external device 20.

The electronics of the drug delivery device 12 comprises a controller 166, a battery 164, and an interface for data exchange with the electronics of the accessory 10, particularly for transmitting data related to drug selection and delivery being stored in an internal memory of for example the controller 166 of the drug delivery device 12.

The interface of the drug delivery device 12 comprises short range wireless communication means 16, particularly an NFC interface circuitry 161 with an NFC loop antenna 162, and/or wired communication means comprising at least one contact 170, particularly a sprung contact, and a serial communication interface circuitry 168 such as an UART/I2C/SPI/1-wire interface circuitry. The short-range wireless communication means 16, particularly the NFC interface circuitry 161 with the NFC loop antenna 162 may be passive, i.e. inductively powered by the electromagnetic field generated by the NFC loop antenna 142 of the accessory. Thus, for wireless data exchange between the accessory 10 and the drug delivery device 12, power supply from the battery 164 may not be necessarily required or can at least be reduced to a minimum.

The controller 166 is coupled to the interface circuitry 168 and the short range wireless communication means 18 and may be configured to control operation of these units such that data for example related to usage of the drug delivery device 12 being stored in the internal memory can be retrieved and transmitted via the short range wireless communication means 16 and/or the wired communication means comprising the interface circuitry 168 to the accessory 10.

According to embodiments, when a data retrieval request is received via either the interface 16 or the contacts 170 and the interface circuitry 168 from the accessory, the controller 166 may retrieve the requested data from the internal memory and control the interface 16 or circuitry 168 to transmit them via the communication link 144, 184 to the accessory 10.

The controller 166 may be configured to protect data related to selected and delivered drug dosages and stored in its internal memory by means of the cryptographic information, for example encrypt the stored data, or control data retrieval requests for the stored data by means of the cryptographic information so that only devices having the cryptographic information may have access to the stored data and may receive the stored data. For example, the controller 166 may be configured to grant only data retrieval requests, which contain the cryptographic information, and reject data retrieval requests without any cryptographic information as unauthorized access.

The electronics of the drug delivery device 12 may be configured to process data retrieval requests from the accessory in an idle, powered off or powered on state. For example, in an idle state, when the controller 166 is switched in a kind of sleep mode if the drug delivery device is not in use, a data retrieval request transmitted from the interface 14 of the accessory may power the wireless interface 16 of the drug delivery device 16, which then may wake-up the controller 166 and power the controller 166 for performing the data retrieval and transmission.

The battery 164 may power the controller 166 and the interface circuitry 168 and the interface 16 and may be for example a lithium button cell, or a rechargeable lithium-ion battery.

According to embodiments, the electronics of the accessory 10 may also be configured to detect a usage of the drug delivery device 12, particularly when a dosage is selected and/or delivered. The controller 30 may upon a usage detection activate the interface 14 to generate an electromagnetic field with the antenna 142 for powering the short-range wireless communication means 16 and particularly also the controller 166. Thus, the electronics of the drug delivery device 12 may be powered by the electromagnetic field generated by the antenna 142 when the accessory 10 detects a usage of the device 12. More specifically, the amount of electrical power supplied via the electromagnetic field can be made sufficient for covering the entire need of the electronic circuitry in the drug delivery device 12 or, alternatively, to only partly contribute thereto. The latter may be found useful when the power consumption of the short-range communication is expected to have significant impact on the lifetime of an eventual internal power source in the drug delivery device 12. In circumstances, covering the additional need may help to make the lifetime of the internal power source lesser dependent on the incidence and total number of data exchanges and/or may allow using a less powerful internal energy source in the drug delivery device.

In a specific embodiment, the drug delivery device 12 may be implemented without the battery 164 and may comprise only electronics such as the NFC interface circuitry 161 and/or the communication interface circuitry 168 and/or the controller 166. The electronics of the drug delivery device 12 may then be inductively powered by the electromagnetic field generated by the NFC loop antenna 142 of the accessory 10 and/or with power supplied via the at least one contact 170 connected to the contact 148 of the accessory 10.

The embodiment without the battery 164 does not require replacing and/or removing of the empty battery 164, which may make usage and disposal of the drug delivery device 12 more easy for patients. Particularly, disposal of the drug delivery device 12 without a battery may be less critical since it usually less harmful to the environment. Another advantage is the storage of drug delivery devices containing drug cartridges particularly at low temperatures, which is usually critical regarding batteries, which may be harmed by a low storage temperature.

A drug delivery device 12 without the battery 164 requires the accessory 10 for data exchange, particularly for recording and storing selected and expelled dosages. The electronics of a drug delivery device 12 without the battery 164 may be provided to detect and/or store different information drug information, particularly information about the drug contained in the device 12 and/or of selected and expelled drug dosages and information on usage of the device 12. For example, information about the drug contained in the device 12 may be stored in the internal memory of the controller 166 or another storage provided in the drug delivery device 12 such as a flash memory of the short-range wireless communication means 16 and/or of the interface circuitry 168. Further information may be stored such as selected and expelled dosages of the drug and usage information about the device 12 as will be described below.

When the accessory 10 is attached to the drug delivery device 12 without the battery 164, the electronics of device 12 may be powered by the accessory 10, and may for example transmit information on the drug to the accessory 10. Further information stored in the device 12 may be also transmitted, for example the date and time of the first usage and last usage of the device 12, the number of usages, dosage related information such as the date, time, amount of the dosages selected and expelled with the device 12.

The drug information may be stored during production of the device 12, while the usage and dosage related information may be stored while and/or after a usage of the device 12 by a patient. The storing of the latter information requires a power supply from the accessory 10, for example be clipping the accessory 10 to the device 12 and activating the accessory 10.

The cryptographic information may be also stored during the production of the device 12, and/or later after production, for example when the drug injection device 12 is a reusable device, which can be used with different drug cartridges. For example, a drug cartridge for usage with the drug delivery device 12 may comprise cryptographic information, e.g. stored in a NFC tag of the cartridge or visibly applied to the cartridge. The accessory 10 may then read the cryptographic information from the cartridge's NFC tag and transmit it to the controller 166 for internally storing in the electronics of the device 12, particularly an internal memory of the controller 166. In case of visibly applied cryptographic information, one of the devices 20, 22 may be used to obtain the cryptographic information and transmit it either directly or via the accessory 10 to the drug delivery device 12.

The powered electronics of the device 12 may cause the controller 166 to execute instructions of a firmware of the device 12, which configure the device 12 to detect selection of a drug dosage and expelling of the selected drug dosage. The detected selected and expelled dosage may then be temporality or permanently stored by the controller 166, particularly in its internal memory, and/or may be transmitted to the accessory 10 immediately after the expelling. The accessory 10 may store the received data in an internal memory and/or transmit it via the communication link 184 to the external device 20. The controller 166 of the drug delivery device 12 and/or the controller 30 of the accessory 10 may also be configured to store any dose related data in its internal storage encrypted with the cryptographic information. Storing dose related data encrypted with the cryptographic information may protect the stored data from unauthorized read access and also may allow to distinguish dose related data receiving from different devices 12, for example when the accessory 10 is provided for usage with different drug delivery devices 12. Thus, a drug mixup of stored dose related data may be prevented, and the accessory 10 can be comfortably used with different drug delivery devices 12.

In addition to the selected and expelled dosages, also time and date and/or usage related information may be stored in the device 12 and/or transmitted to the accessory 10. For example, a counter for the number of usages of the device 12, the time and date of the first and/or last usage of the device 12 may be stored and/or transmitted. The time and date of the first usage and/or last usage may be processed by the accessory 10 to determine the expiration of the drug, for example, when the expiration date after the first and/or last usage of the device is exceeded. The accessory 10 can then issue an alert to the patient informing to replace the drug delivery device 12.

The above described storage and/or transmittal of the drug and dosage related information can be implemented also in a drug delivery device 12 containing the battery 164, i.e. a self-powered drug delivery device 12.

In the following, several embodiments are described with drug delivery devices containing batteries. It should be noted that also drug delivery devices without batteries can be applied instead, provided that the accessories attached to the drug delivery devices are embodied to provide a power supply to the electronics of the drug delivery devices as described above with reference to FIG. 2.

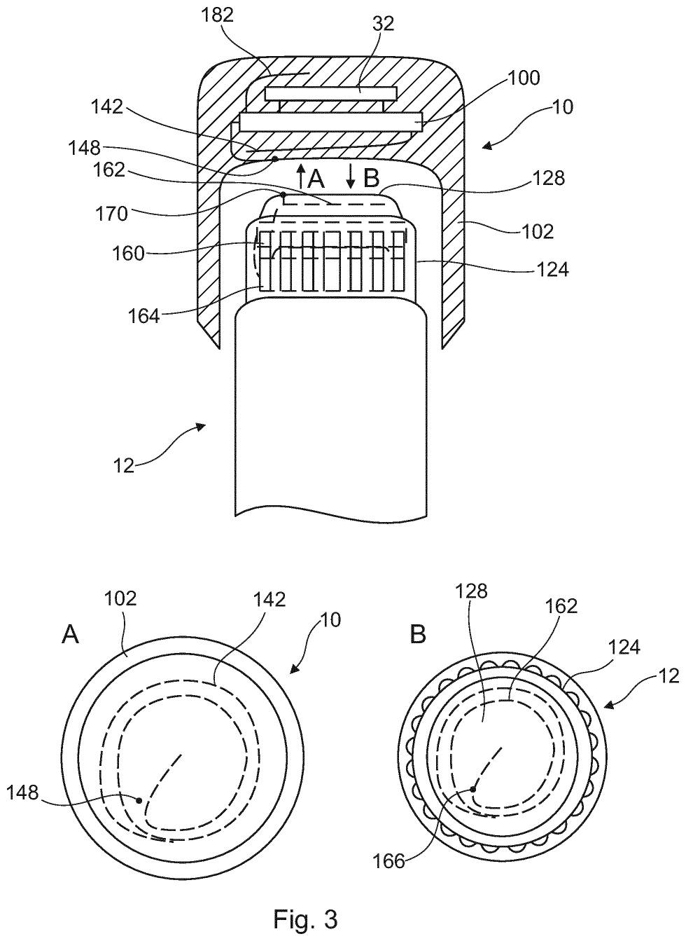

FIG. 3 shows the accessory 10 and its attachment to the drug delivery device 12 in a partly cross-sectional view and shows a view from the bottom on the accessory 10 and a view from the top on the dial knob 124 of the pen 12.

The accessory 10 comprises a cap-like shaped housing 102, which can be pinned on the dial knob 124 of the insulin injection pen 12. The housing 102 may be made of plastics.

Electronics 100 of the accessory 10, which comprises the controller 30 and the circuitries 140, 180, may be integrated in the housing. The electronics 100 may be for example a printed circuit board on which the elements 30, 140, 180 are soldered and wired, a system on a chip, or an integrated circuit.

The NFC loop antenna 142, the antenna 182, the battery 32 for powering the electronics 100 and optionally one or more contacts 148 may also be integrated in the housing 102. The NFC loop antenna 142 and the optional one or more contacts 148 are located in the interior of the housing 102 such that when the housing is pinned on the dial knob 124 an electromagnetic coupling with an NFC loop antenna 162 integrated in the injection knob 128 may be established for data transmission by induction over a short range of some millimetres or one or more centimetres. If the optional one or more contacts 148 and contacts 170 on top of the injection knob 128 are provided, also a wired connection for a wired data exchange can be established. Contacts 148 and/or 162 may be sprung contacts, and/or conductive pads.

An exemplary position of the NFC loop antenna 142 in the housing's 102 interior can be seen in view A and an exemplary position of the NFC loop antenna 162 in the injection knob 128 can be seen in view B (view directions A and B are shown in the partly cross-sectional view). As can be seen from the views A and B, the position of both antennas 142, 162 in the accessory 10 and the pen 12 is such that an electromagnetic coupling for data transmission may be established once the accessory 10 is pinned on the dial knob 142 of the pen 12 with the antennas 142, 162 facing each other with a distance required for electromagnet induction, or in other words when the planes of extension of the loop antennas are arranged in parallel and close to each other for electromagnet induction.

The antenna 182 for long range communication with an external device may be positioned in the top of the housing particularly so that a wireless communication link 182 with the external device 20 can be established, which is unhindered by the battery 32 and the electronics 100 or the like.

A data exchange between the accessory 10 and the injection pen 12 can be initiated by pinning the accessory 10 on the dial knob 124 and powering the electronics 100 of the accessory 10. The data exchange can be accomplished wireless via NFC or wired via an electric contact between the optional contacts.

For a wireless data exchange, the NFC reader circuitry 140 (FIG. 2) comprised by the electronics 100 generates an electromagnetic field with the NFC loop antenna 142. When the distance between NFC loop antennas 142 and 162 is small enough, the electromagnetic field of the antenna 142 may generate by induction an electric current in the antenna 162, which may supply an NFC transceiver circuitry of the electronics 160 contained in the dial knob 124 of the pen 12 (passive NFC circuitry). The NFC transceiver circuitry of the electronics 160 may then transmit data stored in an internal memory of the pen 12 wirelessly via the antenna 162 to the antenna 142 coupled to the NFC reader circuitry 140 particularly according to an NFC communication protocol. The NFC transceiver circuitry of the electronics 160 may also be an active NFC circuitry, which is powered not by an induction current generated in the antenna 162, but by the battery 164 of the pen 10, which is provided for powering the electronics 160 for recording a dosage selection and injection.

For a wired data exchange, the electronics 100 may first detect whether an electric contact via the contacts 148 and 170 exists, particularly by generating via the interface circuitry 150 (FIG. 2), which may be serial communications interface circuitry, which may particularly comprise an UART interface, an I2C bus interface, a Serial Peripheral Interface, or a 1-wire interface, a signal requesting an acknowledgment of the electronics 160 of the pen 12. If an acknowledgment is received from the electronics 160, the interface circuitry 150 may then initiate a communication via the wired connection between the electronics 100 and 160.

The one or more contacts 170 provided on top of the injection knob may be electrically connected to the antenna 162 as shown in the bottom right drawing of FIG. 3, thus allowing to source an electric current directly via the contact(s) 170 in the antenna 162 such that the wired communication means 146 can read a signal modulation by the electronics 160 of pen 12 on the antenna 162.

Particularly, an NFC reader circuitry 150 may be provided and configured to implement a physical layer of a communications protocol layer being adapted for generating the electric current to be sourced in the contact(s) 170 of the antenna 162 of the pen 12. The sourced electric current may generate a signal corresponding to a wirelessly received signal in the antenna 162, which may be processed by the electronics 160 to generate a reply signal to be transmitted via the antenna 162. This reply signal can then be received by the NFC reader circuitry 150, which is able to read the signal modulation on the antenna 162.

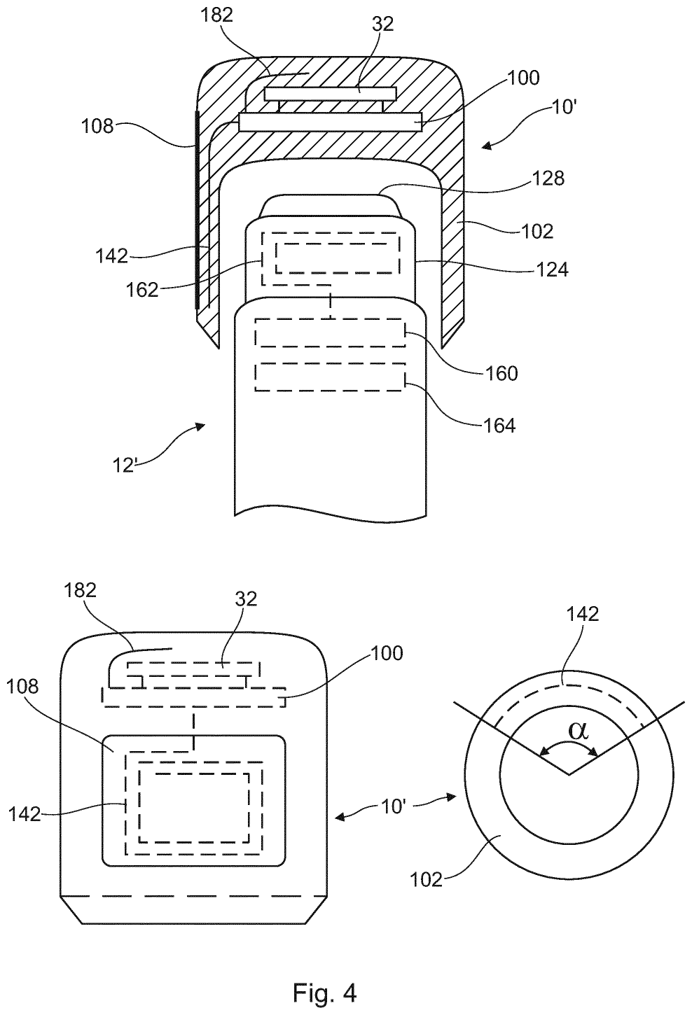

FIG. 4 shows another embodiment of the accessory 10′ and its attachment to the drug delivery device 12′ in a partly cross-sectional view and shows a side view of the accessory 10′ and a view from the bottom on the accessory 10′.

The accessory 10′ also comprises a cap-like shaped housing 102, which can be pinned on the dial knob 124 of the insulin injection pen 12. The housing 102 may be made of plastics.

In this embodiment of the accessory 10′ the NFC loop antenna 142 is located at another position in the housing 102, namely in a flange of the housing 102, which overlaps the dial knob 124 of the pen 12′, when the accessory 10′ is attached to the pen 12′. The position of the NFC loop antenna 142 of the accessory 10′ can be also seen in the bottom drawings in FIG. 4, which show a side view of the accessory 10′ and view from the bottom on the accessory 10′. As can be seen in the view from the bottom on the accessory 10′, the antenna 142 may extend only over a part of the flange's circumference represented by an angle α, which may be between >0° and 360°, particularly as shown in the drawing about 90°. The larger the extension of the antenna, the more power is usually required to generate an electromagnetic field sufficient for data exchange. A smaller the extension of the antenna on the other hand requires a more exact positioning of the antenna 142 and 162 to each other to enable an efficient electromagnetic coupling.

The antenna 162 of the pen 12′ may be positioned in wall of the dial knob 124 of the pen 12′ in order to make electromagnetic coupling between the antenna 142 of the accessory 10′ and the antenna 162 of the pen 12′ more efficient, as shown in the top drawing of FIG. 4. Thus, when the accessory 10′ is attached to the pen 12′ so that the flange of the accessory's housing 102 overlaps the dial knob 142 of the pen 10′, the extension planes of both antennas 142, 162 may be arranged coaxially with a distance allowing an electromagnet coupling between the antennas 142, 162 when both antennas are arranged opposite, i.e. when the antenna 142 extending only over a part of the flange's circumference is positioned nearly opposite to the antenna 162.

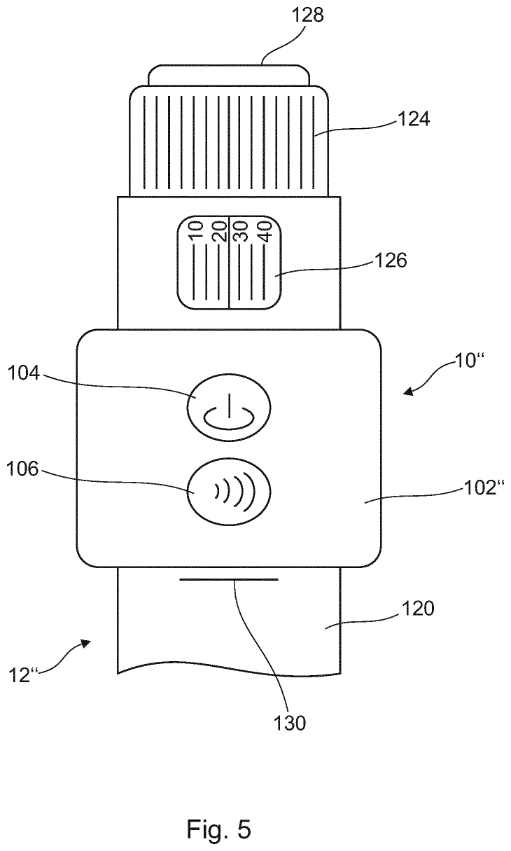

FIG. 5 shows a yet further embodiment of the accessory 10″ and its attachment to the drug delivery device 12″. The accessory 10″ according to this embodiment comprises a sleeve-like shape housing 102″ for pinning on the body 120 of a drug delivery device embodied as an injection pen 12″.

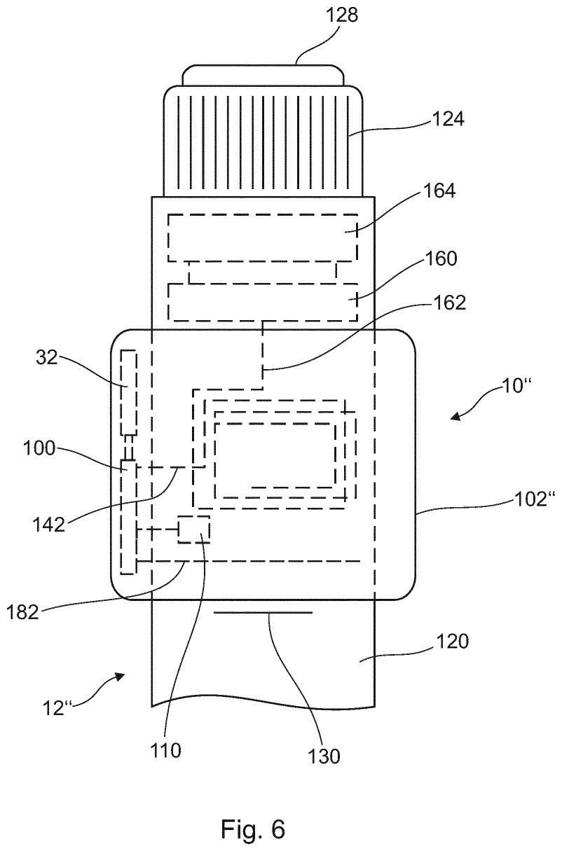

FIG. 6 shows the internal circuitry of the accessory 10″ and of the injection pen 12″ at least in part. The NFC loop antenna 142 of the accessory 10″ is located in the housing 102″, particularly below the inner wall of the housing 102″. The electronics 100 and the battery 32 are also arranged in the housing 102″. Detection means 110 are also provided in the housing 102″ for detecting a usage of the pen 12″. The detection means 110 may be implemented for example by a magnetic switch configured to detect when a dosage is selected with the dial knob 124 and/or a selected dosage is injected by pressing the injection knob 128. The magnetic switch may for example be triggered by a movement of a metallic part of the pen's 12″ internal dosage selection and injection mechanism.

The NFC loop antenna 162 of the pen 12″ may be integrated in a wall of the body 120, particularly at a location below the display 126 (FIG. 5). The electronics 160 and the battery 162 may be arranged close to the dial knob 124, for example being integrated in the dosage selection and injection mechanism (not shown) integrated in the body 120 of the pen 12″. For operation, the accessory 10″ is slipped over the body 120 of the pen 12″, as shown in FIGS. 5 and 6, and may be positioned below the display 126. In such position, the antenna 142 of the accessory 10″ may be located over the antenna 162 of the pen 10″, or at least close to the antenna 162, so that an efficient electromagnetic coupling between the antennas 142 and 162 may be accomplished. Ideally, both antennas 142 and 162 are coaxially arranged on the axis of the body 120.

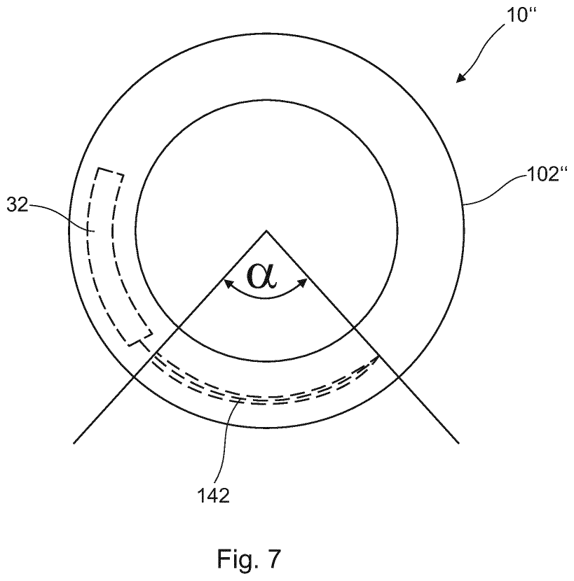

FIG. 7 shows a view on top of the accessory 10″ with the arrangement of the battery 32 and the antenna 142 within the housing 102″. The extension of the antenna 142 in the circumference of the housing 102″ is shown by the angle α, which can be any angle >0° to 360°. In FIG. 7, angle α is about 90° so that a quarter of the cylindrical housing 102″ in circumference contains the antenna 142.

A visual marker 130 can be provided on the body 120 of the pen 12″ indicating the optimal position of the lower end of the housing 102″ of the accessory 10″ for obtaining an efficient coupling of both antennas 142, 162. The electronics 100 may also be configured to assist a user in positioning the accessory 10″ on the body 120, particularly based on a measurement of the inductive coupling of both antennas 142, 162, and for example signal the optimum positioning audible or visible, for example by means of an LED (Light Emitting Diode) integrated in the power button 104.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively, or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia. Examples of DPP4 inhibitors are Vildagliptin, Sitagliptin, Denagliptin, Saxagliptin, Berberine. Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

1-15. (canceled)

16. A system comprising:
a wireless data communication accessory; and
a drug delivery device,
wherein the wireless data communication accessory is configured
to exchange data with the drug delivery device, and
to secure data received from the drug delivery device by using cryptographic information assigned to the drug delivery device.

17. The system of claim 16, wherein the wireless data communication accessory is configured
to communicate with an external device via a wireless communication link, and
to secure the wireless communication link for transmitting the data received from the drug delivery device using the cryptographic information assigned to the drug delivery device.

18. The system of claim 17, wherein at least a part of the cryptographic information is stored in a storage of the drug delivery device and is readable by the external device.

19. The system of claim 16, wherein the wireless data communication accessory is configured
to secure the exchange of data between the wireless data communication accessory and the drug delivery device using the cryptographic information assigned to the drug delivery device.

20. The system of claim 16, wherein at least a part of the cryptographic information is stored in a storage of the drug delivery device and is readable by the wireless data communication accessory.

21. The system of claim 20, wherein the drug delivery device comprises a passive short-range wireless communication device comprising the storage storing the at least a part of the cryptographic information.

22. The system of claim 16, wherein at least a part of the cryptographic information is visibly applied to the drug delivery device.

23. The system of claim 16, wherein at least a part of the cryptographic information is stored in a storage of the external device.

24. The system of claim 16, wherein at least a part of the cryptographic information is stored on a server for downloading.

25. The system of claim 24, wherein the server is a cloud-based server of a cloud storage.

26. The system of claim 16, wherein the exchange of data between the wireless data communication accessory and the drug delivery device requires a pairing of the wireless data communication accessory with the drug delivery device, wherein the cryptographic information is required for the pairing.

27. The system of claim 16, wherein at least one of the drug delivery device or the wireless data communication accessory comprises a storage for storing data related to selected and delivered drug dosages, and wherein the at least one of the drug delivery device or the wireless data communication accessory is configured to protect the stored data related to the selected and delivered drug dosages by using the cryptographic information.

28. A wireless data communication accessory comprising a data exchange interface) for data exchange with a drug delivery device, wherein the wireless data communication accessory is configured to secure data received from the drug delivery device by using cryptographic information assigned to the drug delivery device.

29. The wireless data communication accessory of claim 28, further configured
to communicate with an external device via a wireless communication link, and
to secure the wireless communication link for transmitting the data received from the drug delivery device using the cryptographic information assigned to the drug delivery device.

30. The wireless data communication accessory of claim 28, wherein the data exchange interface is configured to secure the data exchange using the cryptographic information assigned to the drug delivery device.

31. The wireless data communication accessory of claim 28, further configured to read the cryptographic information from a storage of a passive short-range wireless communication device of the drug delivery device by
generating a radio frequency field to power the passive short-range wireless communication device, and
receiving the cryptographic information from the storage of the passive short-range wireless communication device via short-range communication link.

32. A method for securing data from a drug delivery device, the method comprising:
establishing a communication link between the drug delivery device and a wireless data communication accessory;
obtaining, by the wireless data communication accessory, cryptographic information assigned to the drug delivery device;
reading, by the wireless data communication accessory, data from a storage of the drug delivery; and
securing, by the wireless data communication accessory, the read data using the obtained cryptographic information assigned to the drug delivery device.

33. The method of claim 32, wherein establishing the communication link comprises:
pairing the wireless data communication accessory with an external device;
configuring the wireless data communication accessory for communicating with the paired external device; and
securing the communication with the paired external device using the obtained cryptographic information assigned to the drug delivery device, and
wherein the method further comprises transmitting the read data by using the secured communication to the paired external device.

34. The method of claim 32, wherein obtaining the cryptographic information comprises:
generating a radio frequency field for powering a passive short-range wireless communication device; and
receiving the cryptographic information from a storage of the passive short-range wireless communication device via the communication link.

35. The method of claim 32, wherein the communication link is a short-range communication link.
